(19) United States
(12) Patent Application Publication
(10) Pub. No.: US 2011/0136169 A1
(43) Pub. Date: Jun. 9, 2011

(54) EXPRESSION SYSTEM COMPOSITIONS AND METHODS

(75) Inventors: Stephen Anderson, Princeton, NJ (US); Yi Wen Chiang, Somerset, NJ (US)

(73) Assignee: RUTGERS, THE STATE UNIVERSITY OF NEW JERSEY, New Brunswick, NJ (US)

(21) Appl. No.: 12/951,740

(22) Filed: Nov. 22, 2010

Related U.S. Application Data
(60) Provisional application No. 61/263,342, filed on Nov. 20, 2009.

(51) Int. Cl. C12P 21/00; C12N 1/00; C12N 5/00; C12N 15/63

(52) U.S. Cl. 435/69.1; 435/243; 435/325; 435/320.1

(57) ABSTRACT

The present invention relates to novel expression systems and methods for preparing samples for 3D structure determination of a protein based on a protein expression vector, E. coli host, and specific growth media.

### CROSS REFERENCE TO RELATED APPLICATIONS

The instant application claims 35 U.S.C. §119(e) priority to U.S. Provisional Patent Application Ser. No. 61/263,342 filed Nov. 20, 2009.

### STATEMENT REGARDING FEDERALLY SPONSORED RESEARCH OR DEVELOPMENT

This invention was produced in part using funds obtained through grant NIH-NIGMS U54 GM074958 from the National Institutes of Health. The federal government has certain rights in this invention.

### FIELD OF THE INVENTION

The present invention relates to systems and methods for expressing recombinant proteins in a host cell using nucleic acid constructs, wherein the proteins are able to achieve native three-dimensional folding after expression.

### BACKGROUND OF THE INVENTION

Molecular cloning is a well-known and regularly used technique for isolating, replicating, and studying a peptide or protein expression product of a nucleic acid fragment of interest. While cloning methods may be performed in a multitude of different ways, it is most commonly performed by ligating a DNA fragment encoding the protein or peptide into a self-replicating genetic element, e.g. plasmid vector, which is then introduced into a host cell or organism where it is expressed. (See, for example, Sambrook, J. and Russell, D. Molecular Cloning: A Laboratory Manual (Cold Spring Harbor Laboratory Press, New York, ed. 3, 2001.) the contents of which are incorporated by reference herein). At a rudimentary level, such vectors aid in the expression of large quantities of the protein expression product. This provides, inter alia, a useful tool for supplying protein so that its sequence, configuration, three-dimensional structure, etc. may be studied.

A myriad of potential host cells and organisms are available for cloning. Indeed, new hosts and expression vectors for the production of industrially important recombinant proteins are continuously being developed for the purpose of increasing production yields and simplifying down-stream processes. Even still, in many laboratories, Escherichia coli continues to be one of the most frequently employed hosts, in large part, because of its versatility. Current data indicates, however, that many eukaryotic proteins (particularly secretory proteins) are significantly less likely to be expressed and soluble in known Escherichia coli expression systems or otherwise to result in the production of a protein in its native or natural structure configuration. This, of course, drastically reduces the ability of researchers to study the structural biology of the target protein using such expression mechanisms. Despite its success as an expression host, native eukaryotic protein sample production remains as a major challenge to using Escherichia coli-based expression methods.

There is a continuing need in the art for an expression mechanism that can be used to achieve native structural configuration of a protein of interest. There is similarly a continuing need in the art for an expression mechanism that can be used with simple expression hosts, such as Escherichia coli. The instant invention, while not necessarily limited thereto, addresses these needs.

### SUMMARY OF THE INVENTION

In one embodiment, the instant invention relates to a peptide expression system for recombinantly producing a protein in its native configuration comprising:

(a) an expression vector comprising: a staphyloccal protein A promoter region; a secretory signal sequence; at least one staphylococcal protein A IgG-binding domain, and a cloning site for insertion of the protein coding region of interest, wherein the secretory signal sequence, protein A IgG-binding domains, and protein of interest are operably linked to the promoter region to form a single fusion protein when expressed;

(b) a host cell; and

(c) a growth media allowing biosynthetic isotope enrichment of the protein of interest.

In certain embodiments, the staphyloccal protein A promoter region has a nucleotide sequence comprising SEQ ID NO: 3.

In certain embodiments, the secretory signal sequence is derived from Staphylococcus aureus and may encode an amino acid sequence comprising SEQ ID NO: 5.

In certain embodiments, the protein A IgG-binding domain is upstream of the cloning site. It may comprise at least one Z domain and in further embodiments, at least two tandem Z domains. While not limited thereto, in certain embodiments, the Z domain encodes an amino acid sequence comprising SEQ ID NO: 2.

The cloning site for inserting the polynucleotide of interest comprises one or more restriction sites for inserting a polynucleotide of interest therewithin.

The expression vector may also include one or more affinity tag sequences operably linked to the promoter. Such affinity tags include, but are not limited to, a histidine tag, maltose-binding protein, integrin tag, and combinations thereof. In one embodiment, the affinity tag sequence is a histidine tag.

The expression vector may also include a protease cleavage site operably linked to the promoter. Such a protease cleavage site, in certain embodiments, is a site for a protease selected from the group consisting of tobacco etch virus (TEV) protease, PreScission™ protease, Factor Xa protease, trypsin, enterokinase, collagenase, thrombin, and combinations thereof.

The expression vector may also include one or more selectivity markers. Such selectivity markers include, but are not limited to, one or more antibiotic resistance genes.

In certain embodiments, the polynucleotide of interest encodes a protein for a targeted gene.

In further embodiments, the growth media is isotope enriched or otherwise provides for protein labeling. In certain embodiments, the growth media is isotope enriched with one or more amino acid analogs, one non-limiting example of which is selenomethionine. Alternative examples of suitable growth media include, but are not limited to, Celtone™, Spectra-9 Growth Media, and MJ9 media. In further embodiments, the media may include or be derived from any type of growth media, where the media is enriched with isotopic atoms for peptide labeling.

In alternative embodiments, the instant invention relates to a method for recombinantly producing a protein in its native configuration in a host cell comprising:

(a) introducing into the host cell a peptide expression vector comprising:
a staphyloccal protein A promoter region;
a secretory signal sequence;
at least one staphylococcal protein A IgG-binding domain; and
a polynucleotide of interest that is inserted within a cloning site,
wherein the secretory signal sequence, cloning site and protein A IgG-binding domains are operably linked to the promoter region to form a single expression product when expressed; and

(b) culturing the host cell under conditions in which the expression product is expressed and labeled.

In certain embodiments, the staphyloccal protein A promoter region has a nucleotide sequence comprising SEQ ID NO: 3.

In certain embodiments, the secretory signal sequence is derived from Staphylococcus aureus and may encode an amino acid sequence comprising SEQ ID NO: 5.

In certain embodiments, the protein A IgG-binding domain is upstream of the cloning site. It may comprises at least one Z domain and, in further embodiments, a tandem series of at least two Z domains. While not limited thereto, in certain embodiments, the Z domain encodes an amino acid sequence comprising SEQ ID NO: 2.

The cloning site for inserting the polynucleotide of interest comprises one or more restriction sites for inserting a polynucleotide of interest therewithin.

The expression vector may also include one or more affinity tag sequences operably linked to the promoter. Such affinity tags include, but are not limited to, a histidine tag, maltose-binding protein, integrin tag, and combinations thereof. In one embodiment, the affinity tag sequence is a histidine tag.

The expression vector may also include a protease cleavage site operably linked to the promoter. Such a protease cleavage site, in certain embodiments, is a site for a protease selected from the group consisting of TEV protease, PreScission Protease™, Factor Xa protease, trypsin, enterokinase, collagenase, thrombin, and combinations thereof.

The expression vector may also include one or more selectivity markers. Such selectivity markers include, but are not limited to, one or more antibiotic resistance genes.

In certain embodiments, the polynucleotide of interest encodes a protein of a targeted gene.

In further embodiments of the foregoing method, the expression vector may be co-introduced into the host cell with a helper plasmid encoding one or more proteins that aid in folding the expression product into the native configuration of a peptide of the polynucleotide of interest. Such proteins, while not necessarily limited there to, may be selected from the group consisting of DsbA, DsbC, FkpA, SurA, and combinations thereof. In further embodiments, the helper plasmid is pTUM4.

While the expression plasmid may be introduced using any means discussed herein, in certain embodiments, it is introduced into the host cell by transfection. While not limited thereto, the host cell used in the foregoing method may be a prokaryotic host cell. In further embodiments it is a bacterial host cell, and in even further embodiments it is Escherichia coli, such as but not limited to strain RV308. The host cells may be cultured on any media to facilitate cell growth, and, in particular, to facilitate structural characterization of the protein post-purification. In one embodiment, such media includes isotope-enriched amino acids and/or amino acid analogs, which may be provided as a peptide or discrete amino acids. In certain embodiments, the amino acid analogs include selenomethionine. In further embodiments, the media is enriched (and the expression product labeled) with isotopically distinct atoms, such as ²H, ¹³C, ¹⁵N and combinations thereof. Examples of suitable media may be selected from the group consisting of Celtone™, Spectra-9 Growth Media, and MJ9 media. In further embodiments, the media may include or be derived any type of growth media, where the media is enriched with any of the foregoing for peptide labeling.

The foregoing method may further comprise refolding and purifying the expression product using one or a combination of methods discussed herein or otherwise known in the art. The foregoing method may further comprise cleaving the peptide expression of the polynucleotide of interest from the expression product.

In further embodiments, the invention relates to a kit comprising:

an expression vector comprising:
a staphyloccal protein A promoter region;
a secretory signal sequence;
at least one staphylococcal protein A IgG-binding domain, and
a cloning site;
wherein the secretory signal sequence, protein A IgG-binding domains, and cloning site are operably linked to the promoter region to form a single expression product when expressed; and

optionally, a host cell; and

optionally, a growth media allowing specific labeling or biosynthetic isotope enrichment of the protein of interest.

### BRIEF DESCRIPTION OF THE FIGURES

FIG. 1 illustrates a schematic diagram of one embodiment of the expression vector of the instant invention containing an OM3D protein fused directly to a Z domain, but without an affinity tag, protease site or cloning site.

FIG. 2 illustrates a gel profile of expression of the expression vector of FIG. 1 where the host cells were cultured on Celtone, Spectra, MJ9 and 2XTY media and were isolated using osmotic shock or osmotic shock followed by IgG purification.

FIG. 3 illustrates the 2D HSQC spectrum of ¹⁵N-labeled turkey OM3D P1-Lys protein produced using the expression vector of FIG. 1 in Celtone ¹⁵N medium.

FIG. 4 illustrates the construction of the expression vector of the instant invention with a TEV protease cleavage site.

FIG. 5 illustrates a gel profile of osmotic shockates of targets 401, 601, 801/803, & 901/902 in the expression vector of the instant invention.

FIG. 6 illustrates a gel profile of TEV cleaved proteins produced in ¹⁵N enriched medium for 601, 801 & 901 in the expression vector of the instant invention.

FIG. 7 illustrates one embodiment of the expression vector of the instant invention containing a secretion sequence, SpA promoter, two tandem Z domains, and cloning region.

FIG. 8 illustrates cloning sites of the expression vector of the instant invention with and without a histidine affinity tag.

FIG. 9 illustrates the DNA sequence of one embodiment of the vector of the instant invention, Vector pEZZ_TEV. v1.0.

FIG. 10 illustrates the DNA sequence of another embodiment of the vector of the instant invention, Vector pEZZ_TEV. v2.0.

### DETAILED DESCRIPTION OF THE INVENTION

In the following description, terms relating to recombinant nucleic acid technology are used. The following definitions are provided to give a clear understanding of the specification and claims.

The terms “protein” or “peptide” mean a sequence of amino acids of any length, constituting all or a part of a naturally-occurring polypeptide or peptide, or constituting a non-naturally occurring polypeptide or peptide (e.g., a randomly generated peptide sequence or one of an intentionally designed collection of peptide sequences).

The term “fusion” protein means a chimera of at least two covalently bonded polypeptides.

The term “gene” means a nucleic acid (e.g., deoxyribonucleic acid, or “DNA”) sequence that comprises coding sequences necessary for the production of a polypeptide or precursor (e.g., messenger RNA, or “mRNA”). The polypeptide may be a full or partial length coding sequence exhibiting the desired activity or functional properties, e.g. enzymatic activity, ligand binding, signal transduction, etc. The term also encompasses the coding region of a structural gene such that the gene is capable of being transcribed into a full-length mRNA. The term “gene” encompasses both cDNA and genomic forms of a gene. The genomic form or clone of a gene from a eukaryotic cell usually contains the coding region interrupted with non-coding sequences termed “introns” (also called “intervening regions” or “intervening sequences”). Introns are segments of a gene which are transcribed into nuclear RNA (hnRNA); introns may contain regulatory elements such as enhancers. Introns are removed or “spliced out” from the nuclear or primary transcript, and therefore are absent from the mRNA transcript. Introns are rare in genes from prokaryotic cells.

The term “expression” means transcription (e.g., from a gene) and, in some cases, translation of a gene into a protein, or “expression product.” In the process of expression, a DNA chain coding for the sequence of gene product is first transcribed to a complementary RNA, which is often a messenger RNA, and, in some cases, the transcribed messenger RNA is then translated into the gene product—a protein.

The term “operably linked” means that nucleic acid sequences or proteins are operably linked when placed into a functional relationship with another nucleic acid sequence or protein. For example, a promoter sequence is operably linked to a coding sequence if the promoter promotes transcription of the coding sequence. Generally, “operably linked” means that the DNA sequences being linked are contiguous, although they need not be, and that a gene and a regulatory sequence or sequences (e.g., a promoter) are connected in such a way as to permit gene expression when the appropriate molecules (e.g., transcriptional activator proteins—transcription factors—or proteins which include transcriptional activator domains) are bound to the regulatory sequence or sequences.

The term “promoter” means a noncoding DNA sequence usually found upstream (5′ direction) of a gene, providing a site for RNA polymerase to bind and initiate transcription. Transcription of an adjacent gene is initiated at the promoter region. If the promoter is an inducible promoter, the rate of transcription increases in response to an inducing agent.

The terms “vector” or “plasmid” are used in reference to extra-chromosomal nucleic acid molecules capable of replication in a cell and to which an insert target sequence can be operatively linked so as to bring about its replication. Vectors are used to transport DNA sequences into a cell, and some vectors may have properties tailored to produce protein expression in a cell, while others may not. A vector may include expression signals such as a promoter and/or a terminator, a selectable marker such as a gene conferring resistance to an antibiotic, and one or more restriction sites into which insert sequences can be cloned. Vectors can have other unique features (such as the size of DNA insert they can accommodate). A plasmid or plasmid vector is an autonomously replicating, extrachromosomal, circular DNA molecule (usually double-stranded) found mostly in bacterial and protozoan cells. Plasmids are distinct from the bacterial genome, although they can be incorporated into a genome, and are often used as vectors in recombinant DNA technology.

The term “expression vector” means a recombinant DNA molecule containing a desired coding sequence and appropriate nucleic acid sequences necessary for expression of the operably linked coding sequence (e.g., an insert sequence that codes for a product) in a particular host cell.

The terms “transfection,” “transfected,” or “transfect” mean the introduction of foreign DNA into cells (e.g., prokaryotic cells, or host cells). It may be accomplished by a variety of means known to the art, as discussed herein, including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “restriction enzyme” means enzymes (e.g., bacterial enzymes), each of which cut double-stranded DNA at or near a specific nucleotide sequence (a cognate restriction site). Examples include, but are not limited to, EcoRI, RcoRII, BamHI, HindIII, NotI, HincII, HinfI, SmaI, HgiAI, AccI, AhaIII, AluI, ApaLI, BanI, BanII, BglI, ClaI, DraIII, EcoRV, EaeI, HaeI, HaeII, NaeI, NdeI, PstI, PvuI, PvuII, SacI, SalI, ScaI, SpeI, SphI, StuI, XbaI, and XhoII.

The term “restriction site” means a particular DNA sequence recognized by its cognate restriction enzyme.

The terms “to clone,” “cloned,” or “cloning” when used in reference to an insert sequence and vector, mean ligation or recombination of the insert sequence into a vector capable of replicating in a host cell. In this regard, to clone a piece of DNA (e.g., insert sequence), one would insert it into a vector (e.g., ligate it into a plasmid, creating a vector-insert construct) which may then be put into a host so that the plasmid and insert replicate with the host.

The present invention relates to novel expression systems and methods for determining the structural biology of a protein based, in part, on a peptide and protein expression vector derived from the staphyloccal protein A. The vector includes a staphyloccal-derived protein A promoter region, a secretory signal sequence, and one or more staphylococcal-derived protein A IgG-binding domains provided in-frame with a targeted gene or nucleic acid sequence, which is cloned into a desired host cell or organism. These expression systems and methods enable difficult-to-express proteins, such as the extracellular, disulfide-containing domains of mammalian proteins, to be biosynthesized in a recombinant cell, accompanied by proper three dimensional folding within the host. Such proteins can be easily extracted and purified using standard techniques, such as simple osmotic shock procedure, IgG affinity binding, or alternative techniques discussed herein. These expression systems and methods also allow facile labeling of the expressed recombinant proteins by culturing the host cells in media specifically enriched for such purposes. The proteins can then be isolated and purified and its structural biology characterized by one or more of the standard techniques discussed herein or otherwise known in the art.

In one aspect, the vector of the instant invention includes a nucleic acid sequence having a promoter region and one or more protein A IgG-binding domains that are derived from staphylococcal protein A and positioned within the vector upstream of a cloning site. Each of the promoter, IgG binding domains, and the cloning site are operably linked to yield the targeted expression product or fusion protein.

As used herein, the term “staphylococcal protein A” or “SpA” refers to the type I surface protein originally found in the cell wall of the bacteria Staphylococcus aureus. It is encoded by the spa gene and binds proteins from a wide array of mammalian species, most notably IgG antibodies by the domains E, D, A, B, and C. The term “IgG-binding domain” refers to any one or combination of these domains, including combinations or analogues thereof. In certain embodiments, however, the IgG-binding domain refers to one or more Z domains, i.e. engineered analogues of the B domain that also bind to IgG antibodies. These Z domains typically, though not exclusively, contain three alpha helical structures which are arranged in an antiparallel three-helix bundle. While multiple variants of the Z domains are known to exist and are inclusive in the foregoing, in certain embodiments, the Z domain and has a nucleic acid sequence of SEQ ID NO: 1 and an amino acid sequence of SEQ ID NO: 2, as follows:

(SEQ ID NO: 1)
GTAGACAACAAATTCAACAAAGAACAACAAAACGCGTTCTATGAGATCT
TACATTTACCTAACTTAAACGAAGAACAACGAAACGCCTTCATCCAAAG
TTTAAAAGATGACCCAAGCCAAAGCGCTAACCTTTTAGCAGAAGCTAAA
AAGCTAAATGATGCTCAGGCGCCGAAA
(SEQ ID NO: 2)
Val Asp Asn Lys Phe Asn Lys Glu Gln Gln Asn Ala
Phe Tyr Glu Ile Leu His Leu Pro Asn Leu Asn Glu
Glu Gln Arg Asn Ala Phe Ile Gln Ser Leu Lys Asp
Asp Pro Ser Gln Ser Ala Asn Leu Leu Ala Glu Ala
Lys Lys Leu Asn Asp Ala Gln Ala Pro Lys

In certain embodiments of the instant invention, the IgG-binding domains include a Z domain tandem duplication, or two Z domains in juxtaposition and downstream of the promoter region. The IgG-binding domains of the instant invention, again, are not limited to the foregoing Z domain duplication and may include combinations and/or analogues of any of the foregoing.

The SpA promoter region was previously characterized in Gao, J. and Stewart, G. C., J. of Bacteriology, June 2004, p. 3738-3748 and refers to a promoter region derived from the spa gene that is able to express the IgG-binding domain. While multiple variants of the SpA promoter are known to exist and are inclusive in the foregoing, in certain embodiments, the promoter region, including ribosome binding site, has a nucleic acid sequence of SEQ ID NO: 3, as follows:

(SEQ ID NO: 3)
GCGGCCGCTCGAAATAGCGTGATTTTGCGGTTTTAAGCCTTTTACTTCC
TGAATAAATCTTTCAGCAAAATATTTATTTTATAAGTTGTAAAACTTAC
CTTTAAATTTAATTATAAATATAGATTTTAGTATTGCAATACATAATTC
GTTATATTATGATGACTTTACAAATACATACAGGGGGTATTAAT

Again, the instant invention is not limited to the foregoing sequence, however, and may be adapted to include alternative or otherwise known conservative homologues of the foregoing that retain promoter functionality.

Downstream of and operably linked to the promoter region, the vector also includes a signal sequence for extracellular secretion of the expression product in the host cell. While the secretion signal sequence may be any sequence for a eukaryotic or prokaryotic host cell, in certain embodiments, it is for a prokaryotic, particularly a bacterial, host organism. Secretion signals suitable for use in bacterial systems are well known in the art and are generally understood to mean a sequence that translates into about 10 to 30 amino acids, or about 15 to 30 amino acids. It may be provided at the N-terminus of the expressed product, typically upstream of the IgG-binding domains, to enable the expression product to be secreted, i.e. to pass into the bacterial cell periplasmic space. While a wide range of secretory signal signals are known to exist, and are inclusive in the foregoing, in certain embodiments, the secretory signal sequence is compatible with or recognized by a bacterial host organism, particularly Escherichia coli. In further embodiments, the secretory signal sequence is derived from the organism Staphylococcus aureus has a nucleic acid sequence of SEQ ID NO: 4 and an amino acid sequence of SEQ ID NO: 5, as follows:

(SEQ ID NO: 4)
TTGAAAAAGAAAAACATTTATTCAATTCGTAAACTAGGTGTAGGTATTG
CATCTGTAACTTTAGGTACATTACTTATATCTGGTGGCGTAACACCTGC
TGCAAATGCT
(SEQ ID NO: 5)
Leu Lys Lys Lys Asn Ile Tyr Ser Ile Arg Lys Leu
Gly Val Gly Ile Ala Ser Val Thr Leu Gly Thr Leu
Leu Ile Ser Gly Gly Val Thr Phe Ala Ala Asn Ala

Again, the instant invention is not limited to the foregoing sequences and may be adapted to include alternative conservative homologues of the foregoing that retain secretory functionality.

As noted above, the promoter region, secretory signal sequence and IgG-binding domain(s) are all positioned in-frame and operably linked to a cloning site to form a single expression product or fusion protein. Cloning sites are well known to a skilled person and may be obtained readily from the art as suitable. To this end, the cloning site may include any a sequence which facilitates cloning of a gene encoding a protein of interest into the expression system, typically, though not exclusively, through the use of one or more restriction enzyme sites.

The expression vector may also be equipped with one or more additional elements to facilitate post-translational modification and/or protein purification. Such additional elements may include, but are not limited to, affinity tags, cleaveage sites, transcriptional or translational regulatory sites, selective markers, or the like.

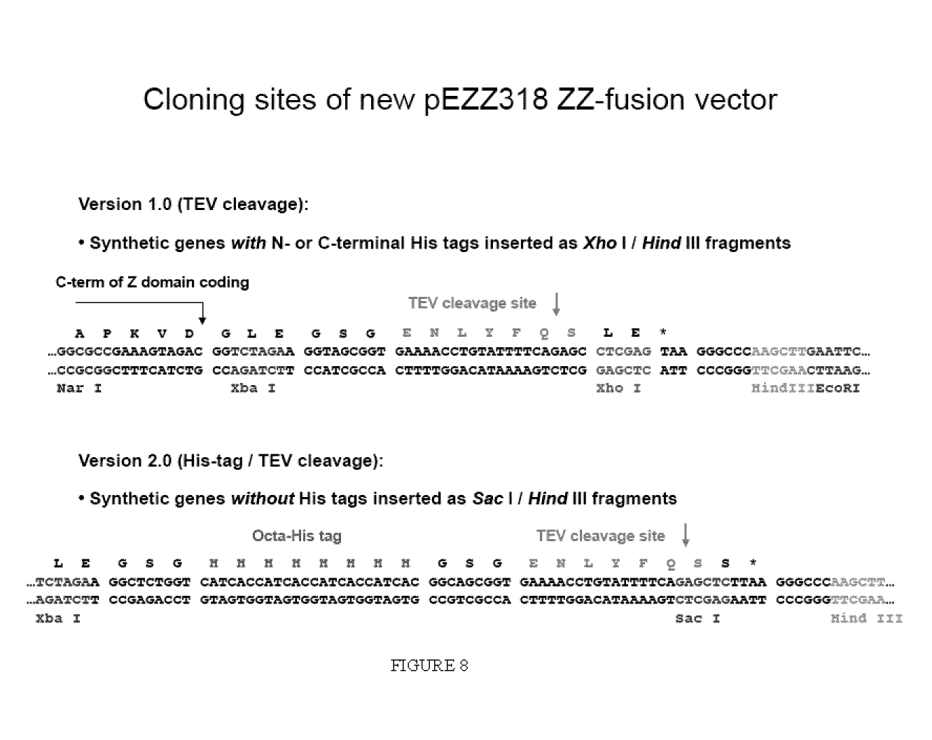

In one embodiment, the affinity tag is a histidine tag that is operably linked to the promoter and positioned at any point down stream thereof. For example, the tag may be positioned at or about the 5′ or 3′ end of the sequence inserted in the cloning site, as illustrated in FIG. 8. In certain embodiments a cleavage site may be inserted between the tag and target sequence, as discussed in greater detail below, to allow for separation of the tag during protein purification.

The instant invention is not limited to a histidine tag and alternative or additional affinity tags may also be provided including, but not limited to, maltose-binding protein, integrin and peptide tags available from commercial suppliers such as Novagen (Madison, Wis.) and Amersham (Piscataway, N.J.). Such affinity tag may be used to isolate or identify the protein using standard techniques well-known to those of skill in the art such as immobilized metal affinity chromatography, western blotting or immunofluorescence.

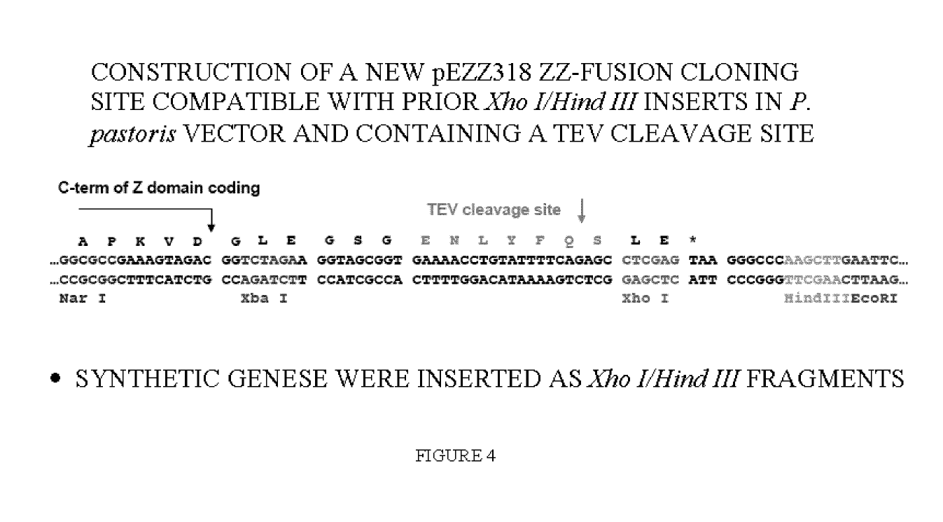

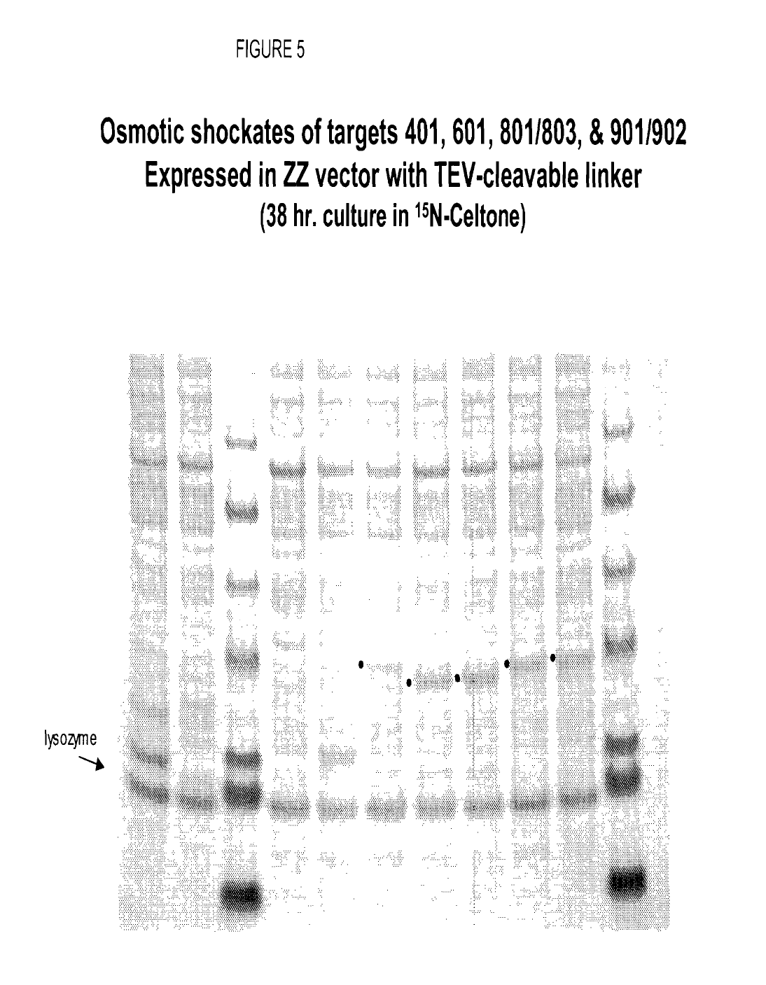

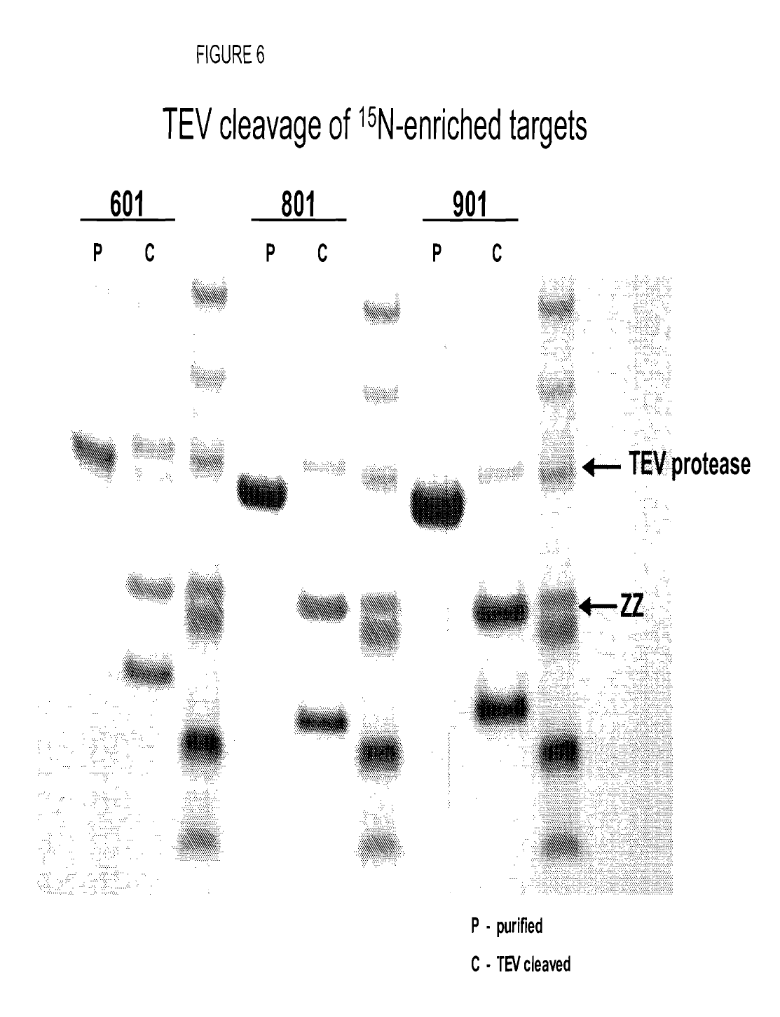

The vector of the instant invention may also include one or more protease sites for post-translational modification and purification of the resulting expression products. Such protease sites may be provided at one or more positions between the target sequence and other components of the expression product or fusion protein, as illustrated in FIGS. 4 and 8. To this end, the expression product may be processed to remove such additional components (e.g. IgG-binding domains, secretory sequence, affinity tags, etc.) in a final processing step. In certain embodiments, the protease cleavage site is recognized by a TEV protease. The instant invention, however, is not limited thereto and may also include one or more alternative sites which may be cleaved by PreScission™ protease, Factor Xa protease, trypsin, enterokinase, collagenase, thrombin, or the like.

The expression vector may also include one or a combination of additional elements suitable for directing expression of the expression product encoded by the expression vector e.g. specific regulatory elements, which facilitate or control expression of the encoded amino acid sequences. Such regulatory elements include, but are not limited to, constitutive; glucose responsive; and/or inducible/regulatable. Regulatory elements herein are selected from regulation sequences and origins of replication (if the vectors are replicated autonomously). Regulation sequences as applicable herein are any elements known to a skilled person having an impact on expression on transcription and/or translation of nucleic acid sequences encoding a fusion protein as defined above and, generally, a protease as defined above. Apart from promoter sequences, such regulation sequences include enhancer sequences, which may lead to an increased expression due to enhanced interaction between RNA polymerase and DNA. Further regulation sequences of expression vectors used herein include transcriptional regulatory and translational initiation signals, so-called “terminator sequences”, etc. or partial sequences thereof.

The expression vector of the instant invention may also contain one or multiple (selection) marker genes for selective expression in in vitro cell cultures. Such (selection) marker genes allow phenotypic selection of transformed host cells and may be provided directly on the expression vector, co-transfected on a second expression vector, or combinations thereof. Selection marker genes may provide prototrophy to an auxotrophic host, biocide resistance (e.g. antibiotic resistance genes) and the like. Examples of selectable markers that confer resistance to the antibiotics include, but are not limited to ampicillin, hygromycin, kanamycin, neomycin, and the like or any selection marker which is not based on antibiotic selection.

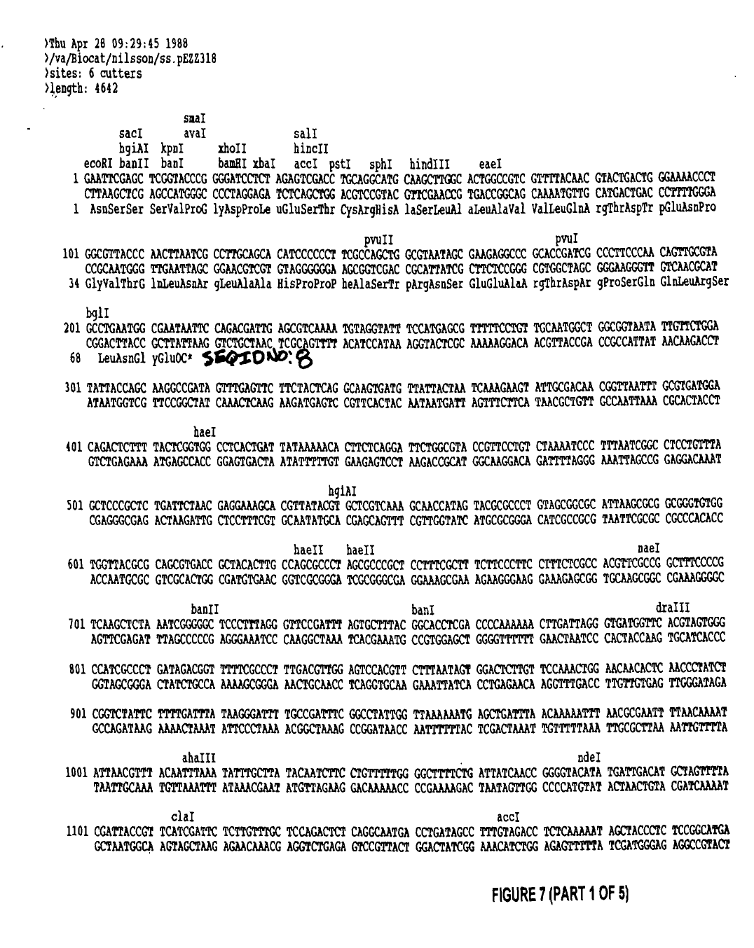

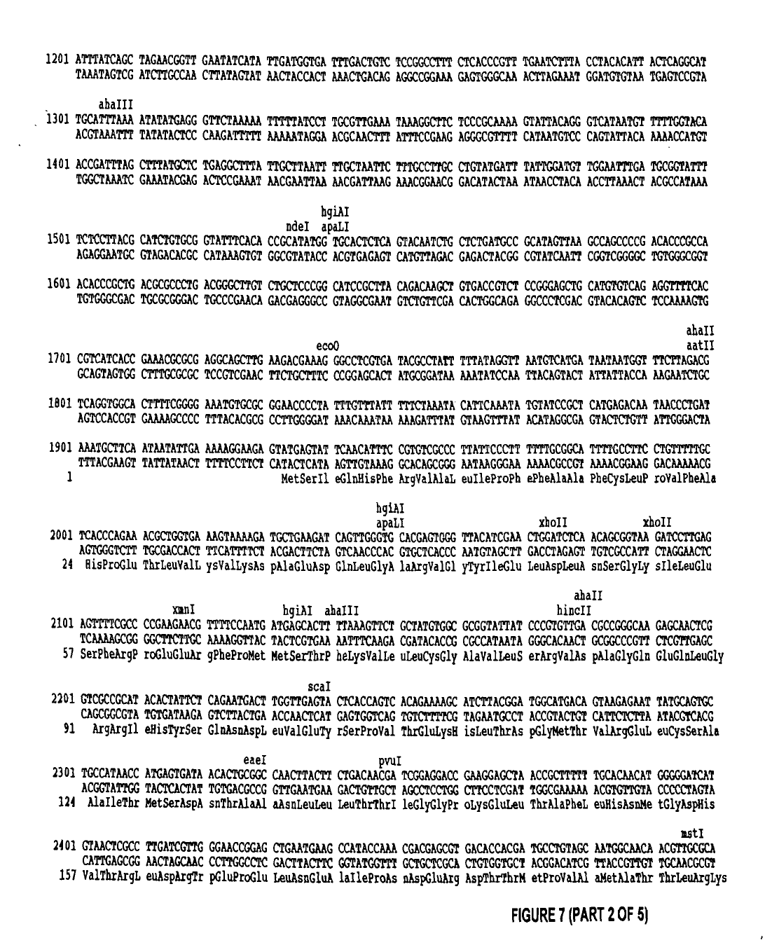

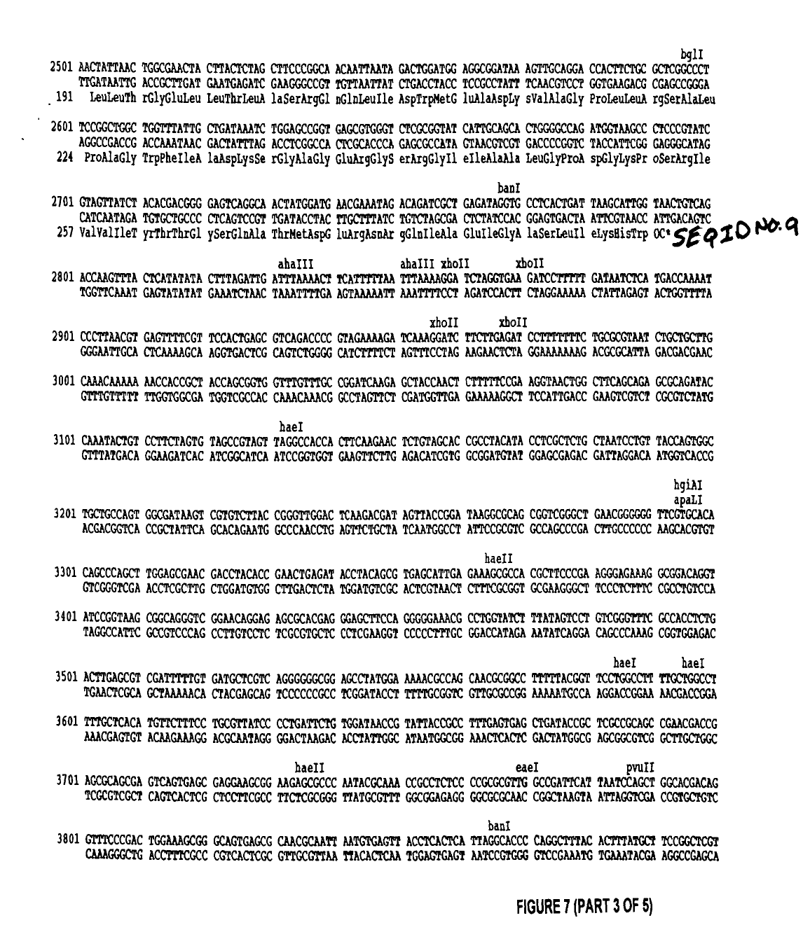

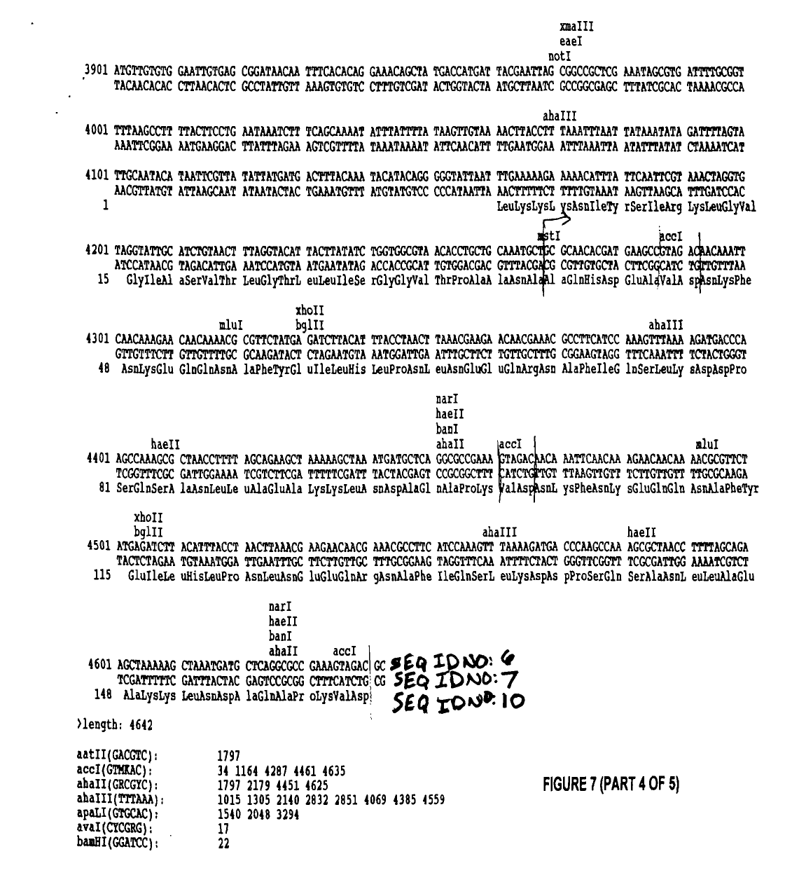

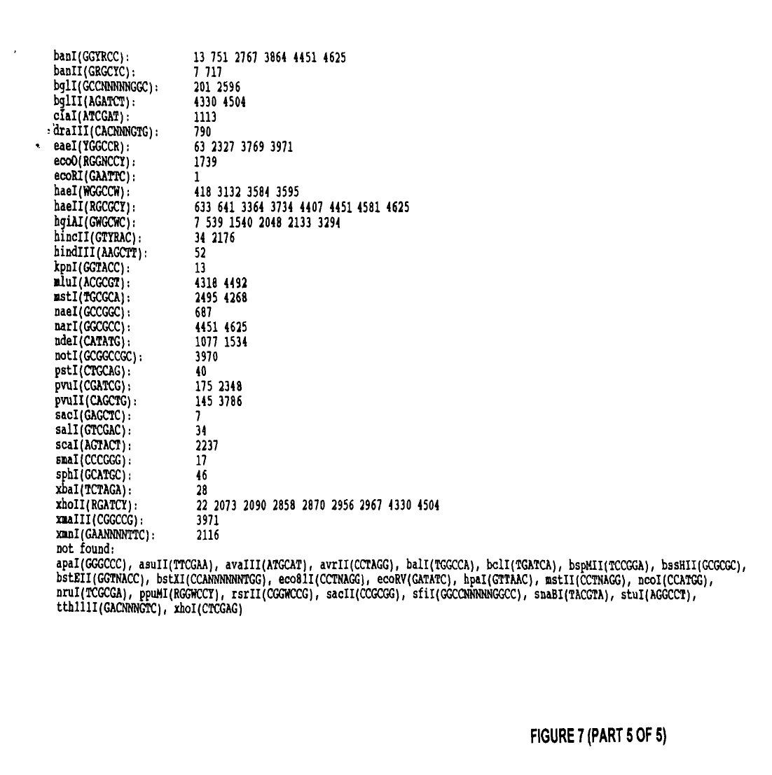

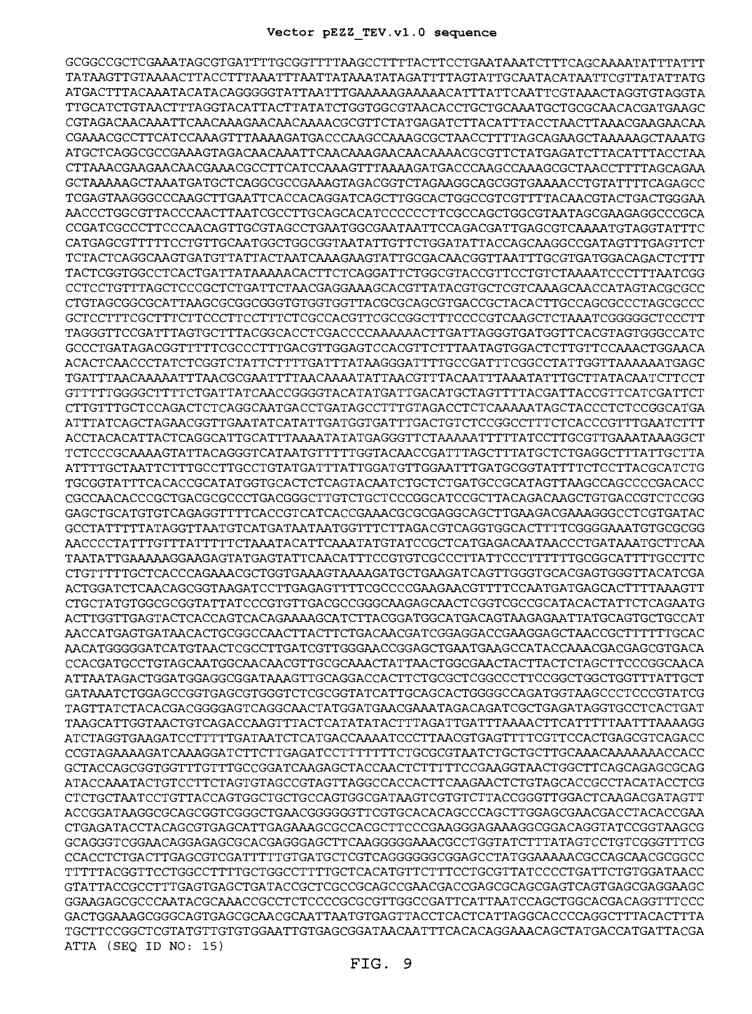

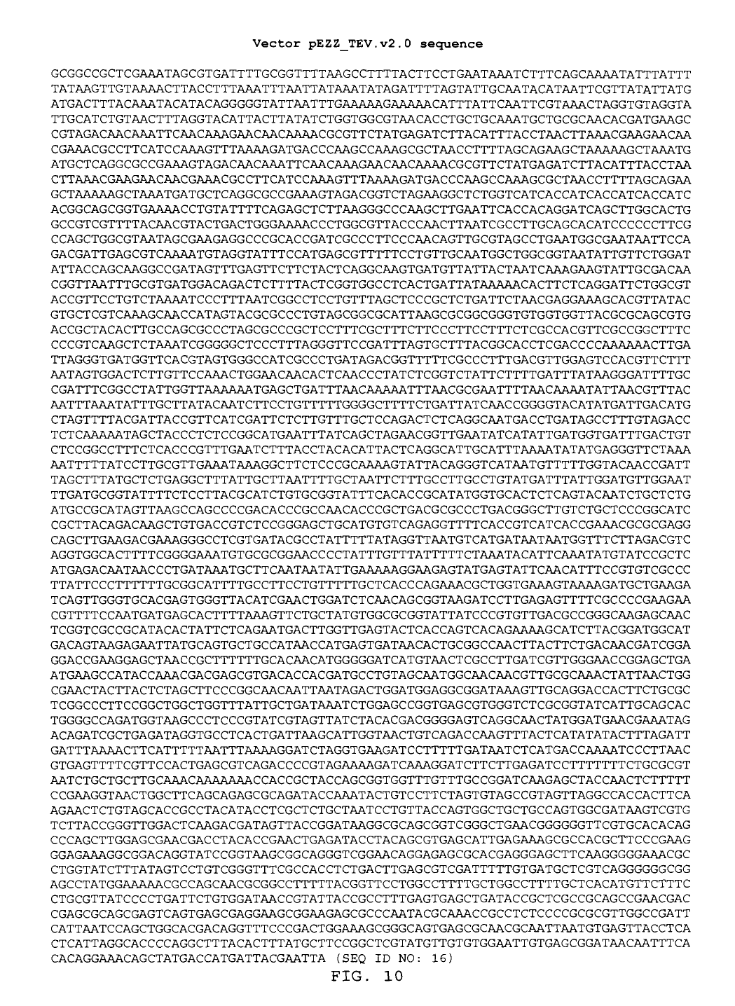

Based on the foregoing, and in one non-limiting aspect of the invention, one embodiment of the expression vector of the instant invention is provided in FIG. 7 and the sense, antisense and amino acid sequences of SEQ ID NOS: 6-10. Alternative embodiments of the expression vector are provided in FIGS. 9 and 10 and SEQ ID NOS: 15-16

The target sequence containing expression vectors, as defined above, may be introduced into a suitable host cell using standard methods known in the art. In a first aspect, the a nucleic acid sequence for the protein of interest is ligated into the cloning region of the foregoing vector. Methods for preparation of nucleic acids are well known in the art and may be applied for preparation of inventive expression vectors by a skilled person as suitable (see e.g. Sambrook et al. 2001) In one example, the nucleic acid of interest may be inserted into the cloning region using one or more of the restriction sites contained therein. Once ligated, the expression vector may be cloned into a host organism.

As indicated above, suitable host cells are to be understood herein as any cell, allowing expression of the expression product or fusion protein and proper folding of the target protein into its native configuration. To this end, any cultivated prokaryotic or eukaryotic host cell, e.g. bacterial, fungal, plant, human and animal host cells, may be used as a host cell. In certain embodiments, the host cell is bacterial host cell and, in even further embodiments, is an Escherichia coli host cell. In certain embodiments the host cell is an Escherichia coli strain RV308 (ATCC 31608).

Transfection methods for introducing an expression vector into the host cell are well understood in the art. (see e.g. Sambrook et al., 2001, supra). Such transfection methods may include, but are not limited to, one or a combination of spontaneous uptake methods, conventional chemical methods (such as the polyamidoamine dendrimer method), the DEAE-dextran method or the calcium phosphate method, physical methods such as microinjection, electroporation, ballistic particle delivery, liposome fusion, lipofection, protoplast fusion, retroviral infection, biolistics, or the like are also contemplated.

The transfected host cell may be cultured on any a suitable culture medium, which may include a transfected cell selection aid (e.g. antibiotic) using standard cell growth protocols known in the art. While not limited thereto, in certain embodiments the cell culture media is any media designed to support the growth and proliferation of a transfected host organism, particularly Escherichia coli. In further embodiments, the culture medium is suitably selected by a person skilled in the art for amino acid labeling of the expression product to facilitate determining the target protein's 3D structure after the protein is purified. In certain embodiments, such culture media is isotope enriched media or includes isotope-enriched amino acids or amino acid analogs provided as discrete units or within a large peptide, which result in a labeled expression product. In certain embodiments, the media is enriched with amino acid analogs such as selenomethionine. In alternative embodiments, the media is enriched (and the expression product labeled) with isotopically distinct atoms, such as ²H, ¹³C, ¹⁵N and combinations thereof. Examples of suitable media may be selected from the group consisting of Celtone™, Spectra-9 Growth Media, and MJ9 media. In further embodiments, the media may include or be derived any type of growth media, where the media is enriched with isotopic atoms for peptide labeling. Thus, standard growth media, such as 2XTY media or any other media known in the art, can be enriched with one or more of the foregoing, as is known in the art.

While not intending to be bound by theory, it is believed that the signal sequence facilitates transportation of the expressed fusion protein through the cellular membrane of the host cell or into the periplasmic space of a bacterial cell where it is folded. To this end, the protein may be folded using native chaperone proteins of the host cell into its proper configuration. The instant invention recognizes, however, not all host cells or organisms are equipped with the requisite chaperone proteins or express such proteins at adequate levels. In such instances, the chaperones may be co-expressed with the instant expression system as additional components to the expression vector above, or, alternatively, on a second “helper” vector. Such chaperones may include any elements commonly known in the art to assist with proper protein folding of the target protein, such as, but not limited to, heat shock proteins, isomerases, or the like. In certain embodiments, the proteins include one or a combination of DsbA, DsbC, FkpA, and/or SurA. In further embodiments, the helper plasmid which may be provided on the helper plasmid pTUM4. (See Schlapschy, M., Grimm, S., and Skerra, A. protein Engineering, Design, & Selection, 19 no. 8 pp. 385-390 (2006) the contents of which are incorporated herein by reference.)

Purification of the peptide is typically achieved using conventional procedures such as osmotic shock, preparative HPLC (including reversed phase HPLC) or other known chromatographic techniques such as gel permeation, ion exchange, partition chromatography, affinity chromatography or counter-current distribution. In certain embodiments, purification may be carried out by means of an antibody. In one example, the antibody is an IgG antibody which recognizes via its Fc domain the Z domain. (See, for example, WO 95/19374, the contents of which are incorporated herein by reference). Additional purification techniques will be readily apparent to one of skill in the art. (see e.g. Sambrook et al. (2001) supra).

Once isolated, the protein of interest may be proteolytically cleaved at the proteolytic cleavage site using a protease as defined above. Proteolytic cleavage releases the protein of interest from the other elements in the expression product, e.g. secretory signal sequence, IgG-binding domain, affinity tag, etc. The protein may then be purified using standard means known in the art or as discussed above. Once purified, the structure of the protein may be characterized according to its physical properties using data from a range of sources, such as spectroscopic techniques, X-ray crystallography, NMR spectroscopy, and alternative techniques known in the art for such purposes.

In further embodiments of the foregoing, the present invention is directed to a kit comprising an inventive expression vector for enhanced expression of a protein of interest as defined above. The kit may contain the inventive expression vector, wherein a nucleic acid sequence containing a cloning site may be substituted with a nucleic acid sequence encoding a protein of interest as defined above. The kit may furthermore contain the inventive expression vector in a suitable host cell as defined herein. Additionally, the kit may contain a manual or instruction guidelines, intended to explain the application of the kit to a skilled person. The kit may be used for enhanced expression of a protein of interest in vitro, or, if applicable, in vivo.

The invention is described more fully by way of the following non-limiting examples. All references cited are hereby incorporated by reference in their entirety herein.

### EXAMPLES

### Example 1

### Construction of Protein A Fusion Vectors pEZZ318_TEV (v1.0) and pEZZ318_His-TEV (v2.0)

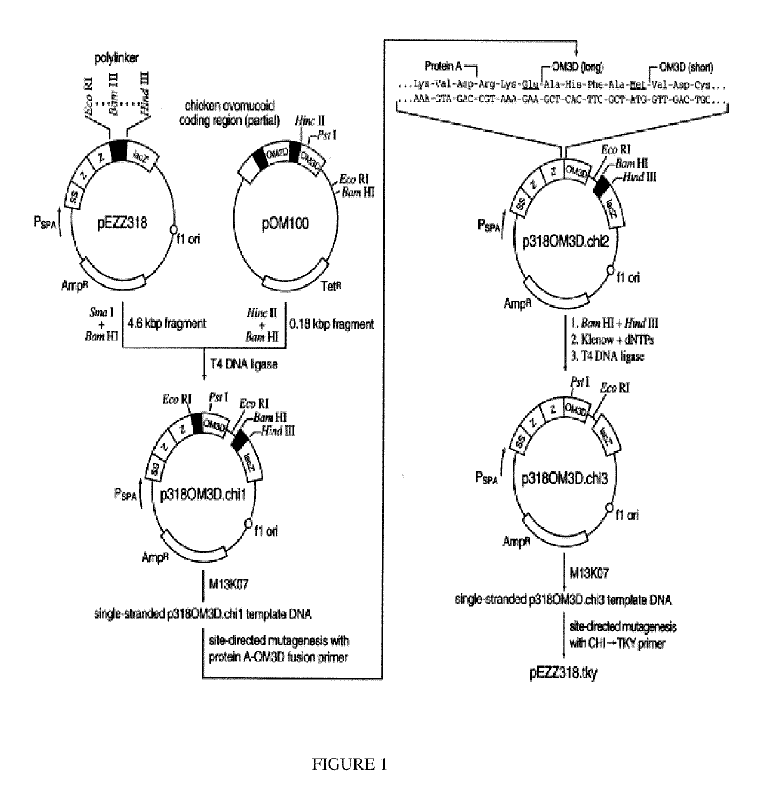

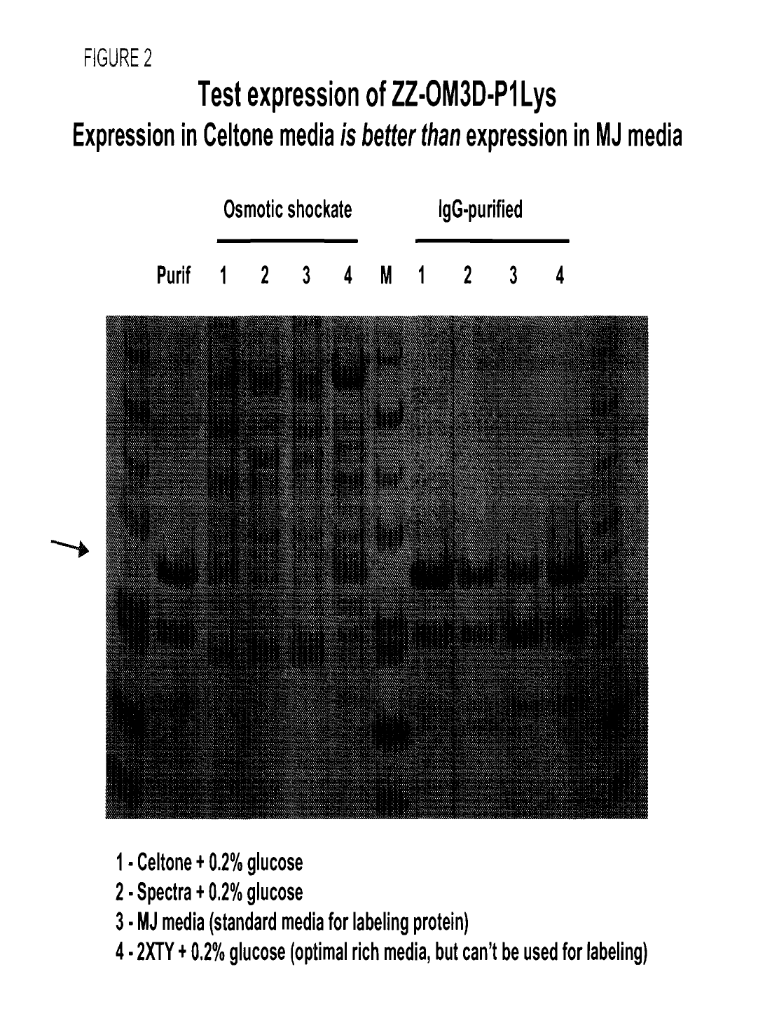

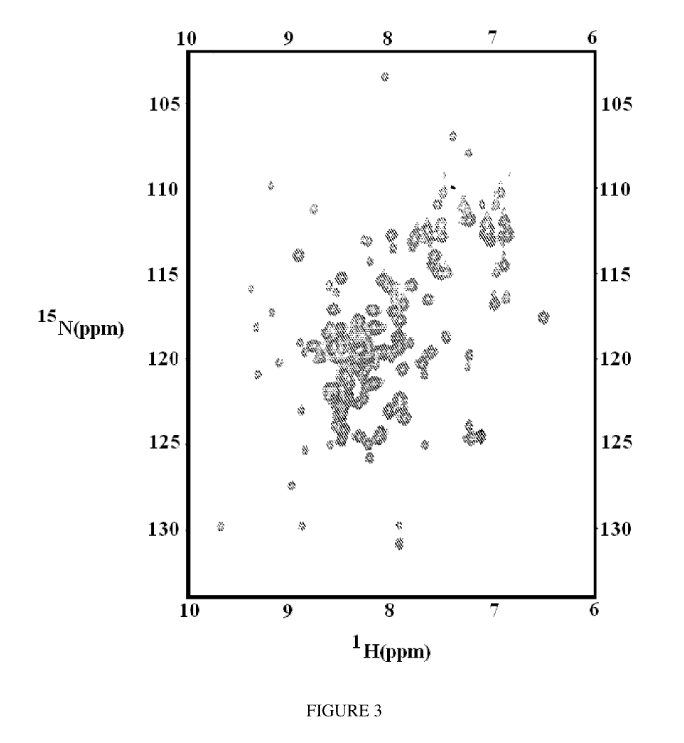

The starting vector was a variant of the pEZZ318 vector expressing a turkey ovomucoid third domain, derived from the chicken ovomucoid pOM100 cDNA clone, as described by Lu et al (1997) J. Mol. Biol. 266, 441-461 (FIG. 9 of this paper is reproduced herein as FIG. 1). Other references: pEZZ318, Nilsson & Abrahmsén (1990) Methods Enzymol. 185, 144-161; pOM100, Stein et al (1978) Biochemistry 17, 5763-5772, and Lai et al (1979) Cell 18, 829-842.

FIG. 7 includes a copy of the sequence of pEZZ318; numbering of nucleotides in the plasmid begins with the G of the Eco RI site at the start of the cloning site polylinker (the last site in the cloning site is the Hind III site, nucleotides 52-57). This polylinker is also represented schematically as a black sector in the pEZZ318 plasmid diagram at the upper left corner of FIG. 9 of Lu et al. (FIG. 1 herein).

Construction of the turkey ovomucoid expression plasmid pEZZ318.tky is discussed in Lu et al (1997) and shown schematically in FIG. 9 from that paper (FIG. 1 herein). The entire cloning site was been replaced in the pEZZ318.tky vector by the coding sequences for turkey ovomucoid third domain as well as the sequences “downstream” of the ovomucoid coding region [up to the BaM HI site in pOM100, including the Eco RI site of this plasmid—see upper left hand portion of FIG. 9 in Lu et al (FIG. 1 herein)].

A new vector was also constructed, pEZZ318_TEV (Version 1.0), deleting all of the ovomucoid coding sequences and “downstream” pOM100 sequences up to the Eco RI site and replacing them with the upper “Version 1.0” sequence shown in FIGS. 8 and 17. This plasmid was designed to allow synthetic genes with N- or C-terminal His-tags to be fused to the upstream synthetic protein A IgG-binding (“Z”) domains by cloning in-frame into Xho I/Hind III-cleaved vector. Separating the ZZ domains from the cloning site is an intervening linker region containing coding sequences for a tobacco etch virus (TEV) protease cleavage recognition site. Thus, treatment of the fusion protein results in cleavage and release of the cloned target (with an additional serine residue—from the serine on the C-terminal side of the cleavage site [see FIG. 8]—attached to the N-terminus of the target. Targets fused to His-tags (at the C-terminus) can thus be purified by metal affinity chromatography both before and after fusion cleavage. The sequence of the resultant vector is given as SEQ ID NO. 15 and is illustrated in FIG. 9.

A second vector was constructed, pEZZ318_His-TEV (Version 2.0), by digesting pEZZ318_TEV (Version 1.0) with Xba I and Hind III and inserting the synthetic Xba I/Hind III fragment shown (lower “Version 2.0” sequence). This fragment codes for a different interdomain linker having a sequence encoding an octameric His-tag upstream of the TEV protease cleavage site (see FIG. 8). Additionally, this linker contains a unique Sac I site overlapping the location of the TEV protease cleavage site. Synthetic genes can thus be cloned into Sac I/Hind III-cleaved vector to produce fusions to the ZZ domains. In this case, although the entire fusion protein can be purified on metal affinity chromatography, the His-tag moiety is cleaved off when the fusion is digested with TEV protease, releasing the cloned target without an attached tag. In this mode, and using a TEV protease which itself has a His-tag, the protease, the ZZ domain, and any uncleaved fusion can be removed simply by passing the digestion mixture through an IMAC column. The sequence of resultant vector is given as SEQ ID NO. 16, and is illustrated in FIG. 10.

### Example 2

### Sample Preparation

The “Version 1.0” expression plasmid containing a sequence encoding the Kazal-1 domain of human follistatin-related protein 3 fused downstream of the ZZ-domains was used to transform E. coli strain RV308. A 2 ml seed culture of this transformant was prepared by growing the cells overnight in ¹³C-, ¹⁵N-labeled Celtone media, supplemented with ¹³C-labeled glucose (0.2%) and 100 μg/ml ampicillin. 1.5 ml of this was then used to inoculate 0.5 L of the sample media in a one liter flask, which was then shaken at 185 rpm for 40 hrs at 30° C. Cells were then centrifuged and the pellet was resuspended in 100 ml of osmotic shock buffer (100 mM Tris-Acetate, 5 mM EDTA, 0.5M sucrose, pH 8.2). Osmotic shockates were prepared by adding equal volumes of ice-cold H₂O to 15 ml aliquots of the resuspended cells, and the suspension was mixed well and incubated on ice for 10 min. Then 0.6 ml of 1M MgSO₄ was added to each 30 ml diluted aliquot and the cells pelleted again by centrifuging at 12,000 rpm for 15 min at 0° C. The supernatant, containing the extruded periplasmic contents of the cells (“osmotic shockate”) was filtered through a 0.45 micron filter. This was then loaded onto a 4 m bed volume Ni IMAC column at 4° C., then this was washed with several column volumes of Ni loading buffer (50 mM Tris-HCl, 500 mM NaCl, 0.02% NaN₃, pH 7.5) containing 15 mM imidazole, and the bound proteins were subsequently eluted with the same buffer containing 250 mM imidazole. Based on the absorbance at 280 nm fractions with significant amounts of protein were pooled and buffer exchanged using a PD10 column TEV buffer (50 mM Tris-HCl, 0.5 mM EDTA, pH 8.0) containing 0.3 mM oxidized glutathione and 3.0 mM reduced glutathione. Then TEV protease was added at a weight ratio of 1:50 TEV protease:fusion protein and the mixture incubated overnight at room temperature to allow the cleavage reaction to take place. The products were then buffer exchanged using PD10 columns into IMAC loading buffer containing 15 mM imidazole and loaded onto a 5 ml by Ni IMAC column. After washing with 5 ml of the same buffer the pass-through and wash fractions were collected and pooled. Samples were then exchanged into various NMR buffers and concentrated. The final yield of purified protein was 5.4 mg.

### Example 3

### Other Targets and pTUM4 Results

Expression (using “version 2.0” of the above vector) was also tested in Celtone media for a number of human targets plus bovine pancreatic trypsin inhibitor (BPTI). Expression was also tested with and without the pTUM4 helper plasmid. The results are shown in Table 1 below. Results are expressed on a roughly quantitative scale of 0 to 5, where “0”=no observable expression and “5”=expression seen in our best media (2XTY+0.2% glucose).

TABLE 1

| NESG ID | Target | no helper | pTUM4 helper |
|---|---|---|---|
| HR6288B | Human FSTL3(E2) | 1 | 3 |
| HR6288C | Human FSTL3(E3) | 4 | 4 |
| HR6288A | Human FSTL3(E4) | 1 | 3 |
| HR6287B | Human FHR1(E3) | 2 | 4 |
| HR6275 | Human FBLN4(E4) | 5 | 5 |
| HR6290B | Human FBLN4(E6) | 1 | 3 |
| HR6290C | Human FBLN4(E7) | 0 (degraded) | 0 (degraded) |
| HR6290D | Human FBLN4(E8) | 1 | 2 |
| HR6276 | Human FBLN4(E9) | 4 | 2 |
| HR6285A | Human PLA2G12B | 0, no ZZ | 0, no ZZ |
| HR6278A | Human TDGF1 | 0 | 0 |
| HR6282A | Human PLA2G5 | 0 | 0 |
| HR6283A | Human FGF21 | 4 | 4 |
| HR6284A | Human IL24 | 0, no ZZ | 0, no ZZ |
| n.a. | BPTI | 1 | 4 |

What is claimed is:

1. A peptide expression system for recombinantly producing a protein in its native configuration in a host cell comprising:
an expression vector comprising:
a staphyloccal protein A promoter region;
a secretory signal sequence;
at least one staphylococcal protein A IgG-binding domains, and
a cloning site for insertion of a protein coding region of interest,
wherein the secretory signal sequence, protein A IgG-binding domains and protein of interest are operably linked to the promoter region to form a single fusion protein when expressed;
a host cell; and
a growth media allowing biosynthetic isotope enrichment of the protein of interest.

2. The peptide expression system of claim 1 wherein the staphyloccal protein A promoter region has a nucleotide sequence comprising SEQ ID NO: 3.

3. The peptide expression system of claim 1 wherein the secretory signal sequence is derived from Staphylococcus aureus.

4. The peptide expression system of claim 1 wherein the secretory signal sequence encodes an amino acid sequence comprising SEQ ID NO: 5.

5. The peptide expression system of claim 1 wherein the protein A IgG-binding domain comprises at least one Z domain.

6. The peptide expression system of claim 1 wherein the protein A IgG-binding domain comprises at least two tandem Z domains.

7. The peptide expression system of claim 1 wherein the protein A IgG-binding domain encodes an amino acid sequence comprising SEQ ID NO: 2.

8. The peptide expression system of claim 1 wherein the protein A IgG-binding domain is upstream of the cloning site.

9. The peptide expression system of claim 1 wherein the cloning site for inserting the polynucleotide of interest comprises one or more restriction sites.

10. The peptide expression system of claim 1 wherein the expression vector further comprises one or more affinity tag sequences operably linked to the promoter.

11. The peptide expression system of claim 10 wherein the affinity tag sequences are selected from the group consisting of a histidine tag, maltose-binding protein, integrin tag, and combinations thereof.

12. The peptide expression system of claim 10 wherein the affinity tag sequence is a histidine tag.

13. The peptide expression system of claim 1 wherein the expression vector further comprises a protease cleavage site operably linked to the promoter.

14. The peptide expression system of claim 13 wherein the protease cleavage site is a site for a protease selected from the group consisting of TEV protease, PreScission™ protease, Factor Xa protease, trypsin, enterokinase, collagenase, thrombin, and combinations thereof.

15. The peptide expression system of claim 1 wherein the polynucleotide of interest encodes a protein.

16. The peptide expression system of claim 1 wherein the expression vector further comprises one or more selectivity markers.

17. The peptide expression system of claim 16 wherein at least one selectivity marker is an antibiotic resistance gene.

18. The peptide expression system of claim 1 wherein the growth media is isotope enriched growth media.

19. The peptide expression system of claim 18 wherein the growth media is isotope enriched with one or more amino acid analogs.

20. The peptide expression system of claim 19 wherein the amino acid analog comprises selenomethionine.

21. The peptide expression system of claim 18 wherein the growth media is enriched with isotopically-distinct atoms selected from the group consisting of ²H, ¹³C ¹⁵N and combinations thereof.

22. The peptide expression system of claim 1 wherein the growth media is selected from the group consisting of Celtone™, Spectra-9 Growth Media, MJ9 media, and 2XTY media.

23. A method for recombinantly producing a protein in its native configuration in a host cell comprising:
introducing into the host cell a peptide expression vector comprising:
a staphyloccal protein A promoter region;
a secretory signal sequence;
at least one staphylococcal protein A IgG-binding domains; and
a polynucleotide of interest that is inserted within a cloning site;
wherein the secretory signal sequence, cloning site and protein A IgG-binding domains are operably linked to the promoter region to form a single expression product when expressed; and
culturing the host cell under conditions in which the expression product is expressed and labeled.

24. The method of claim 23 wherein the staphyloccal protein A promoter region has a nucleotide sequence comprising SEQ ID NO: 3.

25. The method of claim 23 wherein the secretory signal sequence is derived from Staphylococcus aureus.

26. The method of claim 23 wherein the secretory signal sequence encodes an amino acid sequence comprising SEQ ID NO: 5.

27. The method of claim 23 wherein the protein A IgG-binding domain comprises at least one Z domain.

28. The method of claim 23 wherein the protein A IgG-binding domain comprises a tandem series of at least two Z domains.

29. The method of claim 23 wherein the protein A IgG-binding domain encodes an amino acid sequence comprising SEQ ID NO: 2.

30. The method of claim 23 wherein the protein A IgG-binding domain is upstream of the cloning site.

31. The method of claim 23 wherein the cloning site for inserting the polynucleotide of interest comprises one or more restriction sites.

32. The method of claim 23 wherein the expression vector further comprises one or more affinity tag sequences operably linked to the promoter.

33. The method of claim 32 wherein the affinity tag sequences are selected from the group consisting of a histidine tag, maltose-binding protein, integrin tag, and combinations thereof.

34. The method of claim 32 wherein the affinity tag sequence is a histidine tag.

35. The method of claim 23 wherein the expression vector further comprises a protease cleavage site operably linked to the promoter.

36. The method of claim 35 wherein the protease cleavage site is a site for a protease selected from the group consisting of TEV protease, PreScission Protease™, Factor Xa protease, trypsin, enterokinase, collagenase, thrombin, and combinations thereof.

37. The method of claim 23 wherein the polynucleotide of interest encodes a protein.

38. The method of claim 23 wherein the expression vector further comprises one or more selectivity markers.

39. The method of claim 38 wherein at least one selectivity marker is an antibiotic resistance gene.

40. The method of claim 23 further comprising co-introducing to the host cell a helper plasmid encoding one or more proteins that aid in folding the expression product into the native configuration of a peptide of the polynucleotide of interest.

41. The method of claim 40 wherein the one or more proteins that aid in folding expression product are selected from the group consisting of DsbA, DsbC, FkpA, SurA, and combinations thereof.

42. The method of claim 40 wherein the helper plasmid is pTUM4.

43. The method of claim 23 wherein the peptide expression vector is introduced into the host cell by transfection.

44. The method of claim 23 wherein the host cell is cultured on isotope enriched media.

45. The method of claim 23 wherein the expression product is labeled with isotopically-distinct atoms.

46. The method of claim 45 wherein the isotopically-distinct atoms are selected from the group consisting of ²H, ¹³C ¹⁵N and combinations thereof.

47. The method of claim 23 wherein the expression protein is labeled with isotope-enriched amino acids or amino acid analogs.

48. The method of claim 47 wherein the amino acid analog comprises selenomethionine.

49. The method of claim 23 wherein the host cell is cultured on media selected from the group consisting of Celtone™, Spectra-9 Growth Media, MJ9 media, and 2XtY media.

50. The method of claim 23 further comprising purifying the expression product.

51. The method of claim 23 further comprising cleaving the peptide expression of the polynucleotide of interest from the expression product.

52. The method of claim 23 wherein the host cell is a prokaryotic host cell.

53. The method of claim 23 wherein the host cell is a bacterial host cell.

54. The method of claim 23 wherein the host cell is Escherichia coli.

55. The method of claim 54 wherein the host cell is Escherichia coli RV308.

56. A kit comprising:
an expression vector comprising:
a staphyloccal protein A promoter region;
a secretory signal sequence;
at least one staphylococcal protein A IgG-binding domains; and
a cloning site;
wherein the secretory signal sequence, cloning site and protein A IgG-binding domains are operably linked to the promoter region to form a single expression product when expressed;
optionally, a host cell; and
optionally, a growth media allowing specific labeling or biosynthetic isotope enrichment of the protein of interest.


SEQUENCE LISTING (16 sequences)

SEQ ID NO: 1 - DNA, 174 bp, Artificial
  Z domain analogue
gtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaa

SEQ ID NO: 2 - protein, 58 aa, Artificial
  Z domain analogue
VDNKFNKEQQNAFYEILHLPNLNEEQRNAFIQSLKDDPSQSANLLAEAKKLNDAQAPL

SEQ ID NO: 3 - DNA, 191 bp, Artificial
  variant of Staphyloccal protein A promoter region
gcggccgctcgaaatagcgtgattttgcggttttaagccttttacttcctgaataaatctttcagcaaaatatttattttataagttgtaaaacttacctttaaatttaattataaatatagattttagtattgcaatacataattcgttatattatgatgactttacaaatacatacagggggtattaat

SEQ ID NO: 4 - DNA, 108 bp, Artificial
  Staphylococcus aureus secretory signal
ttgaaaaagaaaaacatttattcaattcgtaaactaggtgtaggtattgcatctgtaactttaggtacattacttatatctggtggcgtaacacctgctgcaaatgct

SEQ ID NO: 5 - protein, 34 aa, artificial
  Staphylococcus aureus secretory signal
LKKKNIYSIRKLGIASVTLGTLLISGGVTFAANA

SEQ ID NO: 6 - DNA, 4642 bp, artificial
  Expression vector
gaattcgagctcggtacccggggatcctctagagtcgacctgcaggcatgcaagcttggcactggccgtcgttttacaacgtactgactgggaaaaccctggcgttacccaacttaatcgccttgcagcacatccccccttcgccagctggcgtaatagcgaagaggcccgcaccgatcgcccttcccaacagttgcgtagcctgaatggcgaataattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatttgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttcagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattagcggccgctcgaaatagcgtgattttccggttttaagccttttacttcctgaataaatctttcagcaaaatatttattttataagttgtaaaacttacctttaaatttaattataaatatagattttagtattgcaatacataattcgttatattatgatgactttacaaatacatacagggggtattaatttgaaaaagaaaaacatttattcaattcgtaaactaggtgtaggtattgcatctgtaactttaggtacattacttatatctggtggcgtaacacctgctgcaaatgctgcgcaacacgatgaagccgtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacattcaacaaaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacgc

SEQ ID NO: 7 - DNA, 4642 bp, artificial
  Expression vector
cttaagctcgagccatgggcccctaggagatctcagctggacgtccgtacgttcgaaccgtgaccggcagcaaaatgttgcatgactgacccttttgggaccgcaatgggttgaattagcggaacgtcgtgtaggggggaagcggtcgaccgcattatcgcttctccgggcgtggctagcgggaagggttgtcaacgcatcggacttaccgcttattaaggtctgctaactcgcagttttacatccataaaggtactcgcaaaaaggacaacgttaccgaccgccattataacaagacctataatggtcgttccggctatcaaactcaagaagatgagtccgttcactacaataatgattagtttcttcataacgctgttgccaattaaacgcactacctgtctgagaaaatgagccaccggagtgactaatatttttgtgaagagtcctaagaccgcatggcaaggacagattttagggaaattagccggaggacaaatcgagggcgagactaagattgctcctttcgtgcaatatgcacgagcagtttcgttggtatcatgcgcgggacatcgccgcgtaattcgcgccgcccacaccaccaatgcgcgtcgcactggcgatgtgaacggtcgcgggatcgcgggcgaggaaagcgaaagaagggaaggaaagagcggtgcaagcggccgaaaggggcagttcgagatttagcccccgagggaaatcccaaggctaaatcacgaaatgccgtggagctggggttttttgaactaatcccactaccaagtgcatcacccggtagcgggactatctgccaaaaagcgggaaactgcaacctcaggtgcaagaaattatcacctgagaacaaggtttgaccttgttgtgagttgggatagagccagataagaaaactaaatattccctaaaacggctaaagccggataaccaattttttactcgactaaattgtttttaaattgcgcttaaaattgttttataattgcaaatgttaaatttataaacgaatatgttagaaggacaaaaaccccgaaaagactaatagttggccccatgtatactaactgtacgatcaaaatgctaatggcaagtagctaagagaacaaacgaggtctgagagtccgttactggactatcggaaacatctggagagtttttatcgatgggagaggccgtacttaaatagtcgatcttgccaacttatagtataactaccactaaactgacagaggccggaaagagtgggcaaacttagaaatggatgtgtaatgagtccgtaacgtaaattttatatactcccaagatttttaaaaataggaacgcaactttatttccgaagagggcgttttcataatgtcccagtattacaaaaaccatgttggctaaatcgaaatacgagactccgaaataacgaattaaaacgattaagaaacggaacggacatactaaataacctacaaccttaaactacgccataaaagaggaatgcgtagacacgccataaagtgtggcgtataccacgtgagagtcatgttagacgagactacggcgtatcaattcggtcggggctgtgggcggttgtgggcgactgcgcgggactgcccgaacagacgagggccgtaggcgaatgtctgttcgacactggcagaggccctcgacgtacacagtctccaaaagtggcagtagtggctttgcgcgctccgtcgaacttctgctttcccggagcactatgcggataaaaatatccaattacagtactattattaccaaagaatctgcagtccaccgtgaaaagcccctttacacgcgccttggggataaacaaataaaaagatttatgtaagtttatacataggcgagtactctgttattgggactatttacgaagttattataactttttccttctcatactcataagttgtaaaggcacagcgggaataagggaaaaaacgccgtaaaacggaaggacaaaaacgagtgggtctttgcgaccactttcattttctacgacttctagtcaacccacgtgctcacccaatgtagcttgacctagagttgtcgccattctaggaactctcaaaagcggggcttcttgcaaaaggttactactcgtgaaaatttcaagacgatacaccgcgccataatagggcacaactgcggcccgttctcgttgagccagcggcgtatgtgataagagtcttactgaaccaactcatgagtggtcagtgtcttttcgtagaatgcctaccgtactgtcattctcttaatacgtcacgacggtattggtactcactattgtgacgccggttgaatgaagactgttgctagcctcctggcttcctcgattggcgaaaaaacgtgttgtaccccctagtacattgagcggaactagcaacccttggcctcgacttacttcggtatcatttgctgctcgcactgtggtgctacggacatcgttaccgttgttgcaacgcgtttgataattgaccgcttgatgaatgagatcgaagggccgttgttaattatctgacctacctccgcctatttcaacgtcctggtgaagacgcgagccgggaaggccgaccgaccaaataacgactatttagacctcggccactcgcacccagagcgccatagtaacgtcgtgaccccggtctaccattcgggagggcatagcatcaatagatgtgctgcccctcagtccgttgatacctacttgctttatctgtctagcgactctatccacggagtgactaattcgtaaccattgacagtctggttcaaatgagtatatatgaaatctaactaaattttgaagtaaaaattaaattttcctagatccacttctaggaaaaactattagagtactggttttagggaattgcactcaaaagcaaggtgactcgcagtctggggcatcttttctagtttcctagaagaactctaggaaaaaaagacgcgcattagacgacgaacgtttgtttttttggtggcgatggtcgccaccaaacaaacggcctagttctcgatggttgagaaaaaggcttccattgaccgaagtcgtctcgcgtctatggtttatgacaggaagatcacatcggcatcaatccggtggtgaagttcttgagacatcgtggcggatgtatggagcgagacgattaggacaatggtcaccgacgacggtcaccgctattcagcacagaatggcccaacctgagttctgctatcaatggcctattccgcgtcgccagcccgacttgccccccaagcacgtgtgtcgggtcgaacctcgcttgctggatgtggcttgactctatggatgtcgcactcgtaactctttcgcggtgcgaagggcttccctctttccgcctgtccataggccattcgccgtcccagccttgtcctctcgcgtgctccctcgaaggtccccctttgcggaccatagaaatatcaggacagcccaaagcggtggagactgaactcgcagctaaaaacactacgagcagtccccccgcctcggatacctttttgcggtcgttgcgccggaaaaatgccaaggaccggaaaacgaccggaaaacgagtgtacaagaaaggacgcaataggggactaagacacctattggcataatggcggaaactcactcgactatggcgagcggcgtcggcttgctggctcgcgtcgctcagtcactcgctccttcgccttctcgcgggttatgcgtttggcggagaggggcgcgcaaccggctaagtaattaggtcgaccgtgctgtccaaagggctgacctttcgcccgtcactcgcgttgcgttaattacactcaatggagtgagtaatccgtggggtccgaaatgtgaaatacgaaggccgagcatacaacacaccttaacactcgcctattgttaaagtgtgtccrttgtcgatactggtactaatgcttaatcgccggcgagctttatcgcactaaaacgccaaaattcggaaaatgaaggacttatttagaaagtcgttttataaataaaatattcaacattttgaatggaaatttaaattaatatttatatctaaaatcataacgttatgtattaagcaatataatactactgaaatgtttatgtatgtcccccataattaaactttttctttttgtaaataagttaagcatttgatccacatccataacgtagacattgaaatccatgtaatgaatatagaccaccgcattgtggacgacgtttacgacgcgttgtgctacttcggcatctgttgtttaagttgtttcttgttgttttgcgcaagatactctagaatgtaaatggattgaatttgcttcttgttgctttgcggaagtaggtttcaaattttctactgggttcggtttcgcgattggaaaatcgtcttcgatttttcgatttactacgagtccgcggctttcatctgttgtttaagttgtttcttgttgttttgcgcaagatactctagaatgtaaatggattgaatttgcttcttgttgctrtgcggaagtaggtttcaaattttctactgggttcggtttcgcgattggaaaatcgtcttcgatttttcgatttactacgagtccgcggctttcatctgcg

SEQ ID NO: 8 - protein, 71 aa, Artificial
  Cloning site
NSSSVPGDPLESTCRHASLALAVVLQRTDWENPGVTQLNRLAAHPPFASWRNSEEARTDRPSQQLRSLNGE

SEQ ID NO: 9 - protein, 286 aa, artificial
  Amino acid fragment of expression vector
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 10 - protein, 160 aa, Artificial
  Amino acid fragment of expression vector
LKKKNIYSIRKLGVGIASVTLGTLLISGGVTPAANAAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKVD

SEQ ID NO: 11 - DNA, 82 bp, artificial
  pEZZ318 ZZ-fusion cloning site
ggcgccgaaagtagacggtctagaaggtagcggtgaaaacctgtattttcagagcctcgagtaagggcccaagcttgaattc

SEQ ID NO: 12 - DNA, 82 bp, artificial
  pEZZ318 ZZ-fusion cloning site
ccgcggctttcatctgccagatcttccatcgccacttttggacataaaagtctcggagctcattcccgggttcgaacttaag

SEQ ID NO: 13 - DNA, 88 bp, artificial
  Expression vector cloning site
tctagaaggctctggtcatcaccatcaccatcaccatcacggcagcggtgaaaacctgtattttcagagctcttaagggcccaagctt

SEQ ID NO: 14 - DNA, 88 bp, artificial
  Expression vector cloning site
agatcttccgagacctgtagtggtagtggtagtggtagtgccgtcgccacttttggacataaaagtctcgagaattcccgggttcgaa

SEQ ID NO: 15 - DNA, 4665 bp, artificial
  Vector pEZZ_TEV
gcggccgctcgaaatagcgtgattttgcggttttaagccttttacttcctgaataaatctttcagcaaaatatttattttataagttgtaaaacttacctttaaatttaattataaatatagattttagtattgcaatacataattcgttatattatgatgactttacaaatacatacagggggtattaatttgaaaaagaaaaacatttattcaattcgtaaactaggtgtaggtattgcatctgtaactttaggtacattacttatatctggtggcgtaacacctgctgcaaatgctgcgcaacacgatgaagccgtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacggtctagaaggcagcggtgaaaacctgtattttcagagcctcgagtaagggcccaagcttgaattcaccacaggatcagcttggcactggccgtcgttttacaacgtactgactgggaaaaccctggcgttacccaacttaatcgccttgcagcacatccccccttcgccagctggcgtaatagcgaagaggcccgcaccgatcgcccttcccaacagttgcgtagcctgaatggcgaataattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatttgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttcaagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaatta

SEQ ID NO: 16 - DNA, 4695 bp, artificial
  Vector pEZZ_TEV v2
gcggccgctcgaaatagcgtgattttgcggttttaagccttttacttcctgaataaatctttcagcaaaatatttattttataagttgtaaaacttacctttaaatttaattataaatatagattttagtattgcaatacataattcgttatattatgatgactttacaaatacatacagggggtattaatttgaaaaagaaaaacatttattcaattcgtaaactaggtgtaggtattgcatctgtaactttaggtacattacttatatctggtggcgtaacacctgctgcaaatgctgcgcaacacgatgaagccgtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacggtctagaaggctctggtcatcaccatcaccatcaccatcacggcagcggtgaaaacctgtattttcagagctcttaagggcccaagcttgaattcaccacaggatcagcttggcactggccgtcgttttacaacgtactgactgggaaaaccctggcgttacccaacttaatcgccttgcagcacatccccccttcgccagctggcgtaatagcgaagaggcccgcaccgatcgcccttcccaacagttgcgtagcctgaatggcgaataattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatttgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttcaagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaatta